(19) United States
(12) Patent Application Publication
(10) Pub. No.: US 2025/0092954 A1
(43) Pub. Date: Mar. 20, 2025

(54) Pinch Valve Device

(71) Applicant: SHINANO KENSHI KABUSHIKI KAISHA, Nagano (JP)

(72) Inventor: Koji KODAIRA, Nagano (JP)

(21) Appl. No.: 18/783,652

(22) Filed: Jul. 25, 2024

(30) Foreign Application Priority Data
Sep. 14, 2023 (JP) 2023-148881

(51) Int. Cl. F16K 7/06; F16K 31/04

(52) U.S. Cl. CPC F16K 7/063; F16K 31/047

(57) ABSTRACT

An object is to provide a pinch valve device in which a liquid feeding tube can be easily attached and removed, thus achieving high operability, and in which the compression state of the liquid feeding tube is highly visible. The liquid feeding tube held down to a groove bottom part of the insertion concave groove is squashed in response to that a liquid feeding tube is inserted in an insertion concave groove from an opening by rotationally driving of a stepper motor to cause a second end part to tilt due to the advancement and retreat of a pusher and thus causing a swing lever to swing about a swing shaft, and a first end part swings from a position parallel to the insertion concave groove to a position intersecting the insertion concave groove.

### CROSS-REFERENCE TO RELATED APPLICATION

This application is based upon and claims the benefit of priority of the prior Japanese Patent Application No. 2023-148881, filed on Sep. 14, 2023, and the entire contents of which are incorporated herein by reference.

### TECHNICAL FIELD

The present disclosure relates to a pinch valve device used for, for example, medical equipment, industrial equipment, consumer equipment, and the like.

### BACKGROUND ART

The pinch valve device is used for a blood transfusion device, a dialysis device, and the like, and must clamp as quickly as possible a tube that flows transferred blood into a patient's body, and thus shut off the blood supply to the patient so that air bubbles cannot enter the circulatory system of the body. Alternatively, when feeding a proper amount of liquid agent, the pinch valve device is used to clamp a tube as swiftly and securely as possible in order to prevent a further flow through the tube. The pinch valve device functions as an on/off switch for flow connection between component elements of a fluid system.

The pinch valve device has: a closing element arranged to be movable in an axial direction and having a closing segment to clamp a tube; a permanent magnetic holding device designed to be able to hold the closing element at two different stable equilibrium positions, that is, an opening position and a closing position, by a permanent magnetic force, without needing to supply energy to the permanent magnetic holding device to hold the closing element at each equilibrium position; an electromagnetic actuator device to execute a switching process of moving the closing element from the opening position to the closing position or from the closing position to the opening position, and the like (see PTL 1: JP-A-2023-37586).

### SUMMARY OF INVENTION

### Technical Problem

In the pinch valve device of the foregoing PTL 1, a closing cover that can open and close is provided at an end part in the axial direction of a closed housing. The closing cover, when at an open position, allows the tube to be inserted in a receptacle, and when at a closed position, causes the tube to be pinched and fixed between a tube pinch element and the closing segment.

Since a user needs to carry out the work of opening the closing cover, inserting the tube in the receptacle and then closing the closing cover in this way, the tube attachment work is troublesome. As the pinched state of the tube cannot be visually checked, a setting error may occur. If the tube position shifts, the tube cannot be closed between the tube pinch element and the closing segment.

### Solution to Problem

The present invention has been accomplished in order to solve these problems. An object of the present invention is to provide a pinch valve device in which a liquid feeding tube can be easily attached and removed and does not easily shift in position and in which the compression state of the liquid feeding tube is highly visible.

In order to achieve the foregoing object, a pinch valve device has the following configuration.

The pinch valve device has: a drive source configured to be able to be rotationally driven forward and backward; a pusher configured to advance and retreat in an axial direction through a screw nut screwed on a screw shaft rotationally driven by the drive source; an insertion concave groove in which a liquid feeding tube that is flexible in a direction intersecting a direction in which the pusher advances and retreats is inserted from an opening, in a tube setting block; and a swing lever in which a first end part and a second end part are connected in an L-shape, the first end part being extended to a position intersecting the insertion concave groove, the second end part being linked with the pusher, the first end part swinging about a swing shaft supported by the tube setting block. The liquid feeding tube held down to a groove bottom part of the insertion concave groove is squashed, in response to that the first end part swings from a position parallel to the insertion concave groove to a position intersecting the insertion concave groove by rotationally driving of the drive source to cause the second end part to tilt due to the advancement and retreat of the pusher and thus causing the swing lever to swing about the swing shaft.

Since the insertion concave groove in which the liquid feeding tube can be inserted from the opening is provided in the tube setting block in this way, the work of attaching and removing the liquid feeding tube to and from the tube setting block is easy and the attachment state can be visually checked, and therefore a setting error can be prevented. Also, the liquid feeding tube can be held down to the groove bottom part of the insertion concave groove and squashed and closed by the swing lever, as the drive source is driven to cause the first end part of the swing lever to swing about the swing shaft from the position parallel to the insertion concave groove to the position intersecting the insertion concave groove by the pusher.

Preferably, a pair of lever concave grooves accommodating the first end part of the swing lever may be provided opposite each other in a direction intersecting the insertion concave groove.

Thus, the first end part of the swing lever can be made to swing from a state of being accommodated in a first lever concave groove to an opposite second lever concave groove across the insertion concave groove. As the liquid feeding tube inserted in the insertion concave groove is held down to the groove bottom part of the insertion concave groove and squashed by the first end part, the liquid feeding tube does not escape from the insertion concave groove and high operability is achieved.

If a pair of protruding wall parts where the insertion concave groove is reduced in groove width are arranged opposite each other at both sides of the pair of lever concave grooves, the positioning and retention of the liquid feeding tube can be performed by the pair of opposite protruding wall parts simply by inserting the liquid feeding tube to the groove bottom part of the insertion concave groove.

A pressing surface of the first end part of the swing lever may be tapered.

Thus, since the pressing surface of the first end part of the swing lever is tapered, the liquid feeding tube inserted in the insertion concave groove can be squashed more easily and the liquid feeding can be securely shut off.

A light shielding part may be provided to protrude at the second end part of the swing lever, and a closed state and an open state of the liquid feeding tube may be determined, based on a detection state by a photosensor provided corresponding to the light shielding part.

Thus, since the swing position of the swing lever can be checked via the photosensor, one can carry out work while checking whether the liquid feeding tube is in the closed state or the open state.

### Advantageous Effects of Invention

A pinch valve device in which a liquid feeding tube can be easily attached and removed and does not easily shift in position and in which the compression state of the liquid feeding tube is highly visible.

### BRIEF DESCRIPTION OF DRAWINGS

FIG. 1 is a perspective view of a pinch valve device.

FIG. 2 is a perspective view showing a state where a part of the pinch valve device in FIG. 1 is cut out.

FIGS. 3A, 3B, and 3C are front views showing the pinch valve device before operation, during operation, and after operation, as viewed from the side of an insertion concave groove.

FIGS. 4A, 4B, and 4C are cross-sectional views taken along a direction of arrows Y-Y.

### DESCRIPTION OF EMBODIMENTS

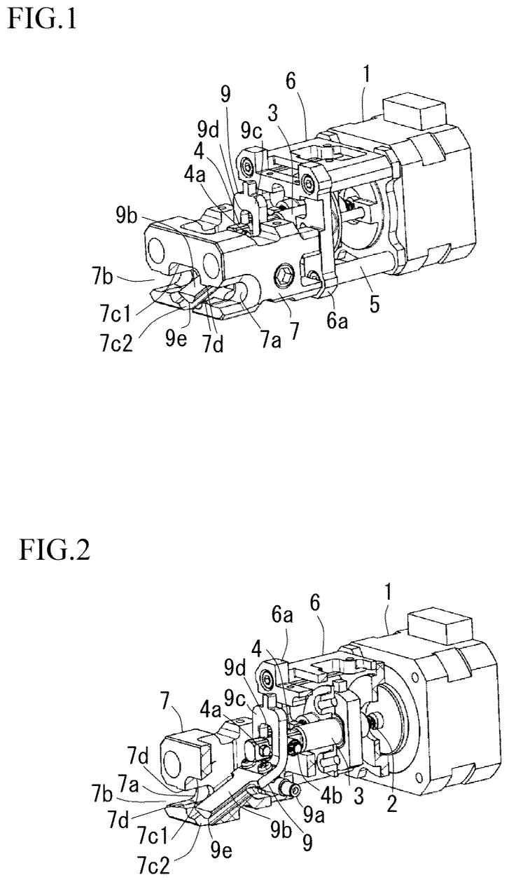

An embodiment of the pinch valve device according to the present invention will now be described with reference to the accompanying drawings of FIGS. 1 to 4.

As shown in FIGS. 1 and 2, a stepper motor 1 is a drive source that can be rotationally driven forward and backward. A screw shaft 2 is coupled to a rotary shaft of this stepper motor 1. A screw nut 3 is screwed on the screw shaft 2. A pusher 4 is assembled in a unified manner with the screw nut 3. The pusher 4 is configured to advance and retreat in an axial direction through the screw nut 3 screwed on the screw shaft 2. At a device main body 6 assembled at an end surface in the axial direction of the stepper motor 1, a plurality of support columns 5 are provided parallel to the screw shaft 2. The support columns 5 support an attachment plate 6a. As the screw shaft 2 rotates, the pusher 4 advances and retreats in the axial direction together with the screw nut 3.

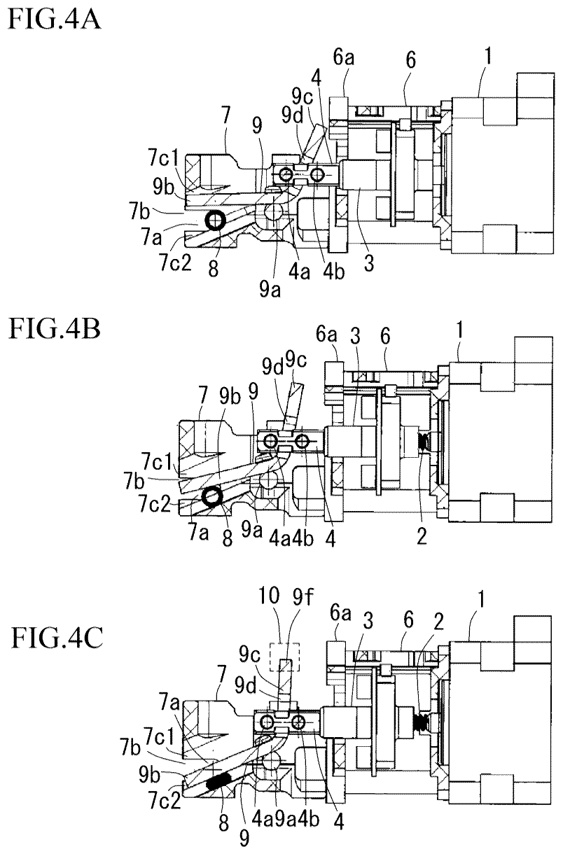

As shown in FIG. 1, a tube setting block 7 is assembled in a unified manner with the attachment plate 6a of the device main body 6. In the tube setting block 7, a U-shaped insertion concave groove 7a in which a liquid feeding tube 8 is inserted in a direction intersecting (orthogonal to) the direction in which the pusher 4 advances and retreats is provided. For the liquid feeding tube 8 (see FIG. 4A) inserted from an opening 7b of the insertion concave groove 7a, a silicone rubber, a resin material or the like that is flexible and is deformed in such a way as to be able to open and close is used.

As shown in FIG. 2, at the tube setting block 7, a swing lever 9 in which a first end part 9b and a second end part 9c are connected in an L-shape is provided in such a way as to be able to swing about a swing shaft 9a. The first end part 9b of the swing lever 9 is extended to a position intersecting the insertion concave groove 7a, and the second end part 9c is linked with the pusher 4. The pusher 4 is inserted in a penetration hole 9d provided in the second end part 9c of the swing lever 9 and is held between a pair of guide shafts 4a, 4b provided to protrude on the pusher 4 at both sides of the penetration hole 9d. When the pusher 4 advances, the second end part 9c comes into contact with the guide shaft 4b and tilts, and the first end part 9b rotates counterclockwise about the swing shaft 9a and squashes the liquid feeding tube 8 in the insertion concave groove 7a and thus closes the liquid feeding tube 8 to disable the feeding of the liquid. When the pusher 4 retreats, the second end part 9c comes into contact with the guide shaft 4a and tilts, and the first end part 9b rotates clockwise about the swing shaft 9a, moves away from the liquid feeding tube 8 in the insertion concave groove 7a, and thus restores the tubular shape to enable the feeding of the liquid.

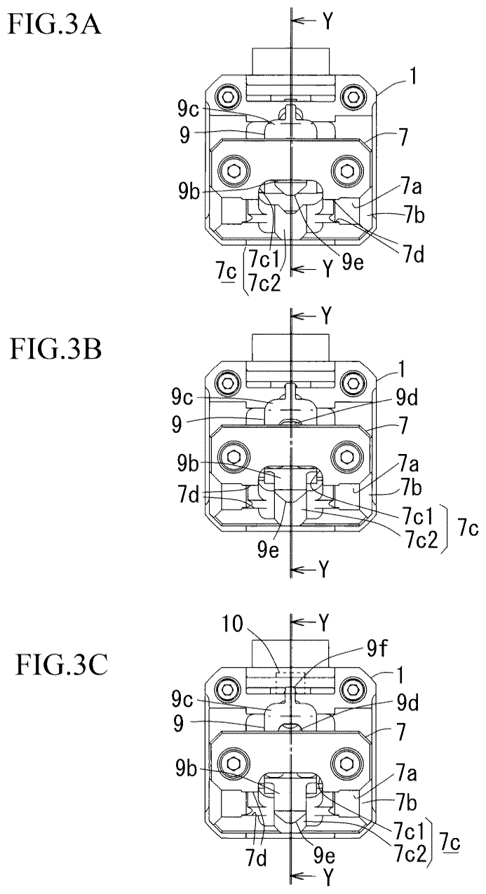

As shown in FIG. 3A, on an inner wall surface of the insertion concave groove 7a of the tube setting block 7, a pair of lever concave grooves 7c (first lever concave groove 7c1, second lever concave groove 7c2) are formed in a direction intersecting (orthogonal to) the insertion concave groove 7a. Of the pair of lever concave grooves 7c, the first lever concave groove 7c1 is a groove to receive the first end part 9b of the swing lever 9 before the liquid feeding tube 8 is attached. The second lever concave groove 7c2 is a groove to receive the first end part 9b when the swing lever 9 squashes the liquid feeding tube 8 inserted in the insertion concave groove 7a.

Near the opening 7b of the insertion concave groove 7a, protruding wall parts 7d that reduce the groove width of the U-shape are arranged opposite each other at both sides of the pair of lever concave grooves 7c. Thus, when the liquid feeding tube 8 is inserted in the insertion concave groove 7a from the opening 7b, the positioning and retention of the liquid feeding tube 8 can be performed by the pair of protruding wall parts 7d provided at both sides of the pair of lever concave grooves 7c.

As shown in FIGS. 2 and 3B, since the first end part 9b of the swing lever 9 compresses the liquid feeding tube 8 so as to squash the liquid feeding tube 8, a pressing surface 9e is tapered to have a wedged cross section. As the pressing surface 9e of the first end part 9b of the swing lever 9 is tapered in this way, the liquid feeding tube 8 inserted in the insertion concave groove 7a can be squashed more easily by the swing lever 9 and the feeding of the liquid can be securely shut off.

The operation of the above-described pinch valve device will now be described with reference to FIGS. 3A to 3C and FIGS. 4A to 4C. In FIGS. 3A to 3C, the illustration of the liquid feeding tube 8 is omitted. FIGS. 3A and 4A show a state where the liquid feeding tube 8 is inserted in the insertion concave groove 7a of the pinch valve device. At this point, the user can attach the liquid feeding tube 8 simply by pushing the liquid feeding tube 8 into the insertion concave groove 7a via the opening 7b and can visually check the tube attachment state. At this point, the pusher 4 is at a retreat position and the first end part 9b of the swing lever 9 is on standby in the first lever concave groove 7c1 intersecting the insertion concave groove 7a (see FIG. 3A).

FIGS. 3B and 4B show a state where the stepper motor 1 of the pinch valve device is activated to cause the pusher 4 to advance. When the stepper motor 1 is rotationally driven in a predetermined direction, the screw shaft 2 rotates and the pusher 4 advances via the screw nut 3. As the pusher 4 advances, the second end part 9c of the swing lever 9 comes into contact with the guide shaft 4b and tilts, and the first end part 9b rotates counterclockwise about the swing shaft 9a and the first end part 9b starts to squash the liquid feeding tube 8 in the insertion concave groove 7a. At this point, the first end part 9b is in contact with the liquid feeding tube 8 in the state of being inclined to be high on the side of the groove bottom part of the insertion concave groove 7a and low on the side of the opening 7b, and rotates about the swing shaft 9a while holding down the liquid feeding tube 8 toward the groove bottom part of the insertion concave groove 7a from the opening 7b. Therefore, the liquid feeding tube 8 does not escape and come off from inside the insertion concave groove 7a.

FIGS. 3C and 4C show a state where the pusher 4 of the pinch valve device is made to advance so as to squash the liquid feeding tube 8 in the insertion concave groove 7a and shut off the feeding of the liquid. The second end part 9c of the swing lever 9 tilts while still in contact with the guide shaft 4b. The first end part 9b rotates further counterclockwise about the swing shaft 9a, and the pressing surface 9e of the first end part 9b enters the second lever concave groove 7c2 intersecting the insertion concave groove 7a and squashes the liquid feeding tube 8. Thus, the liquid feeding tube 8 is elastically deformed and the feeding of the liquid is shut off.

In the case of starting the feeding of the liquid through the liquid feeding tube 8, the stepper motor 1 is rotationally driven backward to cause the pusher 4 to retreat via the screw nut 3. Thus, the second end part 9c of the swing lever 9 comes into contact with the guide shaft 4a and tilts, and the first end part 9b rotates clockwise about the swing shaft 9a and the first end part 9b moves away from the liquid feeding tube 8 in the insertion concave groove 7a and returns to the first lever concave groove 7c1 intersecting the insertion concave groove 7a (see FIGS. 3A and 4A). When the first end part 9b moves away, the liquid feeding tube 8 restores the tubular shape due to its own elasticity and is enabled to feed the liquid.

Since the insertion concave groove 7a in which the liquid feeding tube 8 can be inserted from the opening 7b is provided in the tube setting block 7 in this way, the liquid feeding tube 8 can be easily attached to and removed from the tube setting block 7 and the attachment state can be visually checked. Also, since the stepper motor 1 is driven to cause the pusher 4 to make the first end part 9b of the swing lever 9 swing about the swing shaft 9a from the position parallel to the insertion concave groove 7a to the position intersecting the insertion concave groove 7a, the swing lever 9, while swinging, holds down the liquid feeding tube 8 toward the groove bottom part of the insertion concave groove 7a. Therefore, the liquid feeding tube 8 does not escape from the insertion concave groove 7a and high operability is achieved.

As indicated by a dashed line in FIGS. 3C and 4C, a light shielding part 9f may be provided to protrude at the second end part 9c of the swing lever 9, and the closed state and the open state of the liquid feeding tube 8 may be determined, based on the detection state by a photosensor 10 provided corresponding to the light shielding part 9f.

Thus, since the swing position of the swing lever 9 can be checked via the photosensor 10, one can carry out work while checking whether the liquid feeding tube 8 is in the closed state or the open state.

The above-described pinch valve device can be broadly applied to a manufacturing site where a liquid agent is fed by a fixed quantity each time, or a dialysis device, a blood transfusion device, and the like as medical equipment.

What is claimed is:

1. A pinch valve device comprising:
a drive source configured to be able to be rotationally driven forward and backward;
a pusher configured to advance and retreat in an axial direction through a screw nut screwed on a screw shaft rotationally driven by the drive source;
an insertion concave groove in which a liquid feeding tube that is flexible in a direction intersecting a direction in which the pusher advances and retreats is inserted from an opening, in a tube setting block; and
a swing lever in which a first end part of an L-shaped lever member is extended to a position intersecting the insertion concave groove, a second end part is linked with the pusher, and the first end part swings about a swing shaft supported by the tube setting block, wherein
the liquid feeding tube held down to a groove bottom part of the insertion concave groove is squashed, in response to that the first end part swings from a position parallel to the insertion concave groove to a position intersecting the insertion concave groove by rotationally driving of the drive source to cause the second end part to tilt due to the advancement and retreat of the pusher and thus causing the swing lever to swing about the swing shaft.

2. The pinch valve device according to claim 1, wherein
a pair of lever concave grooves accommodating the first end part of the swing lever are provided opposite each other in a direction intersecting the insertion concave groove.

3. The pinch valve device according to claim 2, wherein
a pair of protruding wall parts where the insertion concave groove is reduced in groove width are arranged opposite each other at both sides of the pair of lever concave grooves.

4. The pinch valve device according to claim 1, wherein
a pressing surface of the first end part of the swing lever is tapered.

5. The pinch valve device according to claim 1, wherein
a light shielding part is provided to protrude at the second end part of the swing lever, and a closed state and an open state of the liquid feeding tube is determined, based on a detection state by a photosensor provided corresponding to the light shielding part.
